(19) United States
(12) Patent Application Publication
(10) Pub. No.: US 2020/0093628 A1
(43) Pub. Date: Mar. 26, 2020

(54) ANKLE SUPPORT AND METHOD FOR SECURING THE SAME

(71) Applicant: Ossur Iceland ehf, Reykjavik (IS)

(72) Inventors: Sindri Pall SIGURDSSON, Reykjavik (IS); Bjorn OMARSSON, Reykjavik (IS); Tomas NJALSSON, Reykjavik (IS); Harry Duane ROMO, Foothill Ranch, CA (US); Liza A BOON, Reykjavik (IS)

(21) Appl. No.: 16/582,157

(22) Filed: Sep. 25, 2019

Related U.S. Application Data
(60) Provisional application No. 62/736,023, filed on Sep. 25, 2018.

(51) Int. Cl. A61F 5/01; A43B 7/20; A43B 7/14

(52) U.S. Cl. CPC A61F 5/0127; A43B 7/144; A43B 7/20

(57) ABSTRACT

An ankle support includes a sleeve and a heel strap that offers comfortable and secure heel fit. The ankle support has a versatile strap system that can be easily configured for different indications and forms of restriction. The strap system accounts for the anatomy of the ankle by offering stretch where needed to provide effective motion control while balancing comfort. The strap system has segments lacking elasticity or stretch to stably secure the strap system about a user's ankle while accommodating the anatomy of the ankle and minimizing migration and discomfort.

### FIELD OF THE DISCLOSURE

The disclosure relates to the field of orthopedic devices, and more particularly to ankle supports and methods of securing the same.

### BACKGROUND

Ankle sprains are frequent injuries in athletes and often happen during physical activities, such as running and jumping. Ankle ligaments, muscles and tendons, and the bones of the foot and ankle, create the internal supports needed to keep an ankle safe from injury. These internal supports are important links from the foot to the hip, allowing for ideal coordination and performance.

Taping and ankle braces are often used as interventions to prevent ankle injuries and may reduce the risk of recurrent ankle sprains. Ankle braces can provide an individual with proprioceptive stimulation, which offers improved proprioception and sensory feedback. While braces differ in design, material, and movement restriction of the ankle-foot joint complex (e.g., semirigid and lace-up braces), the goals are to provide sufficient protection but also sufficient flexibility of the ankle during sports and activities of daily living.

Ankle braces serve as external supports to limit certain motions, such as plantar flexion/inversion (movement at the ankle joint that points the foot downward away from the leg and turns the foot inward) and provide awareness of where the ankle joint is in space. It has been found that for maximum effectiveness, ankle braces should fit comfortably in the shoes one wears during an activity, which also helps with compliant brace use.

Ankle braces may be easy to apply and do not require or offer much in customization, aside from adjustment in size or tightening. Known ankle braces may be used for different ankle issues yet may be limited to the extent they can control or limit ankle movement.

Some ankle braces are too restrictive in preventing movement of a user's ankle, or are not sufficiently restrictive because they insufficiently prevent movement of a user's ankle, particularly in certain directions. Known ankle braces include plastic or metal components that limit breathability, are bulky and uncomfortable, and lack compression for proprioception. Many known ankle braces inconsistently apply pressure or support over portions of a user's ankle, or insufficiently anchor straps to the brace, creating discomfort, poor fitting, and migration over the ankle. There is a problem of existing braces not offering consistent comfort, breathability and bulkiness while securing of the ankle brace is not maintained consistently during repeated use. Further, because most ankle braces are purchased off the shelf, the configuration of immobilizing or restricting elements of the brace may be poorly suited to a particular user's dynamic needs.

Another problem of known ankle braces is that users needing ankle stabilization—in contrast to ankle rehabilitation or prevention of major injury—may be poorly served by the customarily bulky and heavy-duty injury-prevention ankle braces common in the market, and by rehabilitation braces. Ankle stabilization may be required for users who have recovered from past injuries, suffer from various disabling pathologies, or require long-term assistance. Such users may not require rigid struts and supports to prevent a major injury or rehabilitation-centric solutions that severely limit particular motions based on damage to a torn ligament, for example. Rather, such users need for a comfortable, lightweight, and customizable ankle support may be poorly served and overlooked by the existing market for injury-prevention and rehabilitation ankle braces.

As a user may have sustained need for ankle brace, it is helpful that the ankle brace is simple and intuitive to don. Known ankle braces may involve a plurality of straps to secure the brace to the ankle, and therefore require knowledge and incur time for donning. The plurality of straps or lack of means for consistent or versatile donning, such as adapting the brace for different stages of support and/or treatment, may lead to further injury or deter the user from properly wearing the brace. There is a problem of versatility in ankle braces and ready adaptation according to changing user needs.

Taping, by contrast, is a customized process and can be designed for a specific athlete, sport, and/or instabilities. However, for sustained use, an individual may not have ready access to a clinician that regularly can customize and repeatedly perform taping of the ankle. Once taping is completed, a user rarely has flexibility to properly adapt the taping based on the user's dynamic needs, for example during different stages of recovery from an injury or in response to changes in swelling or the user's weight. It has been a challenge for braces to appropriately mimic taping techniques, especially when applied to a broad spectrum of users and their anatomies. There is a problem of balancing the benefits of taping in an ankle brace for individual and dynamic, adjustable stabilization.

For at least these reasons, there is a need for an improved ankle brace that overcomes the shortcomings of known ankle braces and taping practices, such as lack of customizability and difficulty of properly and consistently donning, while merging the benefits of known ankle braces and tapings.

### SUMMARY

An ankle support according to embodiments provided herein mimics taping configurations while offering versatility combined with a compressive sleeve. The ankle support is flexible, breathable and lightweight for use throughout the day and during activity. The ankle support is low-profile (to fit within a user's shoe, for example) and has a mesh structure to provide ventilation.

The ankle support can be used for ankle-injury rehabilitation while permitting varied ranges of motion during rehabilitation—for sprained lateral or medial ligaments, for example. The ankle support also provides arch and ankle joint stability, with the strap system arranged for motion restriction including eversion and inversion control.

The ankle support has a heel strap that offers comfortable and secure heel fit, whereas it is often found in known ankle braces that the heel is ignored or not focused on. The arrangement of the ankle support offers proprioception due to the compressive nature of the sleeve, and due to the arrangement of the strap system on the compressive sleeve. The sleeve and strap system conform closely to the user's anatomy, leading to more comfortable, sustained, compliant and consistent use by users.

The ankle support has a versatile strap system that can be easily configured by a user and/or a clinician for different indications and forms of restriction. The strap system accounts for the anatomy of the ankle by offering stretch where needed to provide effective motion control while balancing comfort. Likewise, the strap system has segments lacking elasticity or stretch to stably secure the strap system about a user's ankle while accommodating the anatomy of the ankle and minimizing migration and discomfort.

By providing an ankle support according to embodiments of the disclosure comprising a lightweight and breathable sleeve, a versatile strap system, and/or a heel strap, the problems of existing ankle supports, including ankle braces and taping practices, providing ineffective and/or cumbersome assistance as discussed above are addressed.

These and other features, aspects, and advantages of the present disclosure will become better understood regarding the following description, appended claims, and drawings.

### BRIEF DESCRIPTION OF THE DRAWINGS

The ankle support embodiments disclosed in the following drawings are illustrated for example only. The elements and combinations of elements described below and illustrated in the drawings can be arranged and organized differently to result in embodiments still within the spirit and scope of the ankle support embodiments described herein.

FIG. 1 illustrates a perspective view of an embodiment of an ankle support according to the present disclosure.

FIG. 2 illustrates an elevational view of a first side of an ankle support according to another embodiment of the present disclosure.

FIG. 3 illustrates an elevational view of a second side of the ankle support in FIG. 2.

FIG. 4 illustrates an elevational view of an ankle support according to another embodiment of the present disclosure with the strap system in an extended configuration.

FIG. 5 illustrates a schematic view of a rear side of the ankle support in FIG. 1.

FIG. 6 illustrates a schematic view of the first side of the ankle support in FIG. 1 in three strapping configurations.

### DETAILED DESCRIPTION OF VARIOUS EMBODIMENTS

The embodiments of the disclosure, namely an ankle support and methods for using the same, are adapted for a human body, and may be dimensioned to accommodate different types, shapes, and sizes of human body sizes and contours. For explanatory purposes, the ankle support embodiments described herein correspond to different sections of a body and are denoted by general anatomical terms for the human body.

The embodiments of the ankle support are particularly referred to as corresponding to anterior and posterior sides defined by an anterior-posterior plane. The embodiments are referred to likewise as corresponding to lateral and medial sides defined by a median plane, and to dorsal and plantar sides of a foot. Proximal and distal sections of the ankle support are defined according to the normal understanding of proximal (nearer to the center of the body) and distal (situated away from the center of the body relative to proximal). The anatomical terms described are not intended to detract from the normal understanding of such terms as readily understood by one of ordinary skill in the art of orthopedics and human anatomy.

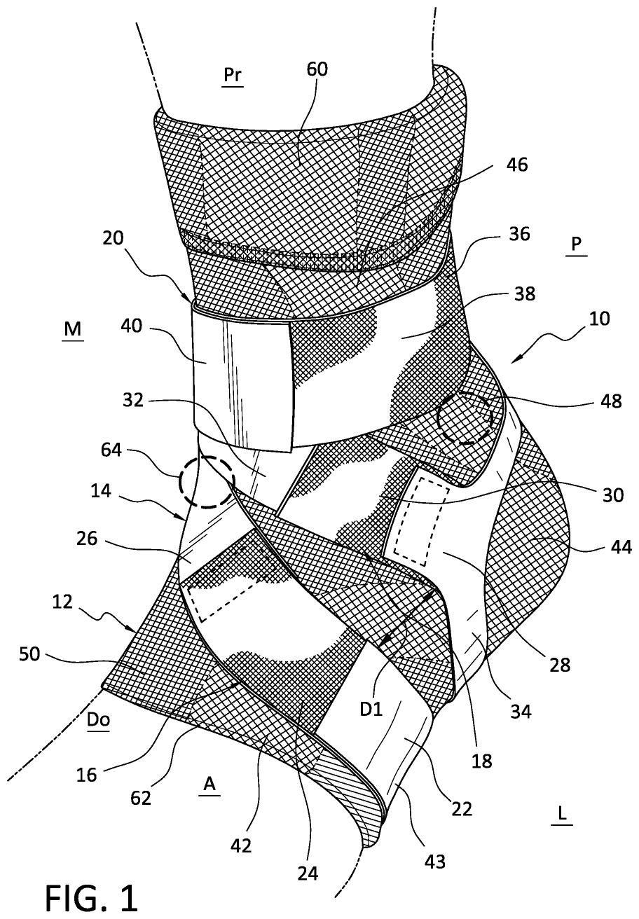

FIG. 1 illustrates an embodiment of an ankle support 10 having first and second sides divided by a median plane. The embodiment shows the first side as being on the lateral side L and the second side as being on the medial side M. It will be understood that components of the ankle support 10 may be reconfigured on opposite sides. While the ankle support is described as suitable for either a right or left ankle and foot, thereby not being universal in fitting to accommodate both right and leg ankles, the ankle support may be adapted for universal fitting.

The ankle support 10 includes a compressive sleeve 12 and a strap system 14. The strap system 14 has a first strap subassembly 16 anchored to and extending from the first side L of the ankle support 10. A second strap subassembly 18 is anchored to and extends from the first side L of the ankle support 10 and is spaced apart from the first strap subassembly 16. The first and second strap subassemblies 16, 18 join at a third strap subassembly 20, at junction 64. The third strap subassembly 20 is elongate and has a free end 57 securable to at least one of the first, second or third strap subassemblies 16, 18, 20. The third strap subassembly 20 is wrappable about and over the sleeve 12, preferably near or about a proximal end (Pr) of the sleeve 12. Such a position is above a user's malleolus and may extend into a lower leg area about a user's tibia and fibula (i.e., lower calf and shin), providing stabilization and injury prevention.

As the sleeve 12 is preferably formed from an elastic knit fabric, the sleeve 12 delivers stretch and compression for pain relief and proprioception. The sleeve 12 may be alternatively or in addition constructed from other materials that sufficiently compress a user's ankle, such as Neoprene or other elastomers, for example.

The sleeve 12 preferably has a tubular shape, which combined with its inherent elasticity, offers easy donning of the ankle support 10. The elastic knit fabric yields a less bulky ankle support compared to known ankle braces, such as those with stirrups or a plurality of rigid and semi-rigid components. The sleeve 12 allows the ankle to move without significant restriction and allows for adaptation of the restriction of the ankle according to the degree the strap system 14 is secured. The sleeve 12 allows for freedom of movement and comfort, while comfortably accommodating a variety of shoes. The sleeve 12 also provides greater comfort by preventing the straps of the strap system 14 from pressing directly into a user's skin.

The sleeve 12 preferably provides medical-grade compression, such as between 20 mm Hg-30 mm Hg. The yarns used to form the sleeve 12 may include Nylon and Clastokine (rubber). Other yarn constructions may be used, including Elastane (spandex) instead of Clastokine. The sleeve 12 may have gradual compression, being greatest at the foot, and lowest above the ankle. The compression of the sleeve 12 may drop at the edges, so an edge of compression is avoided, reducing discomfort on a user's skin. The sleeve 12 may be modified so different regions have different compressibility and stretchability properties, which may be achieved by a knitting process or a secondary process, such as by heating or fusing the yarns in predetermined areas to provide greater support or pressure relief.

For example, the sleeve 12 may comprise regions of different elasticities as beneficial for different portions of anatomy. For example, a first section 42 located generally at a distal portion or aspect of the foot may be configured to be more elastic than a second section 46 located generally at or above the ankle, which may be less elastic to prevent over-extension of the ankle in certain directions. The first section 42 may be less elastic than a third section 50 located generally at a dorsal portion or aspect of the foot which may be configured to allow flexion more easily. The elasticities may be defined by the knit patterns of the sleeve 12 or by the materials used to form the respective sections 42, 46, 50 and may have different aesthetic properties.

The shape of the sleeve 12 is form-fitted to the general shape of an ankle, which enables the ankle support 10 to be consistently donned so it matches the strap system 14 in correspondence to portions of the foot and ankle. The shape reduces migration, and the sleeve 12 may be provided with anti-migration means, such as those described in U.S. patent application publication no. 2018/0078398, published on Mar. 22, 2018, and incorporated herein by reference.

The sleeve 12 may have gripper elements for anti-migration means, such as tacky silicone dots, on predetermined locations inside the sleeve 12, aside from just a proximal end to reduce likelihood of the sleeve 12 shifting relative to the user's skin and changing a level of ankle stabilization. Anti-migration means may also or alternatively be provided on the exterior of the sleeve 12, such as along a plantar portion, to allow the user to use the ankle support without shoes.

The first, second, and third sections 42, 46, 50 of the sleeve 12 define at least first and second elasticity gradients 42, 46, 50 having different elasticities from one another. The elasticity gradients 42, 46, 50 may be arranged and formed in a similar manner as taught in U.S. patent application publication no. 2018/0078398. The elasticity gradients 42, 46, 50 may correspond to how the strap system 14 extends over the sleeve 12 and may be arranged according to elastic and inelastic segments of the strap system 14, and anatomical portions of a foot and ankle, to provide stretch where needed for comfort, and lack of stretch in areas susceptible to injury and/or to restrain movement. The different elasticities may further be provided based on different and dynamic compression needs.

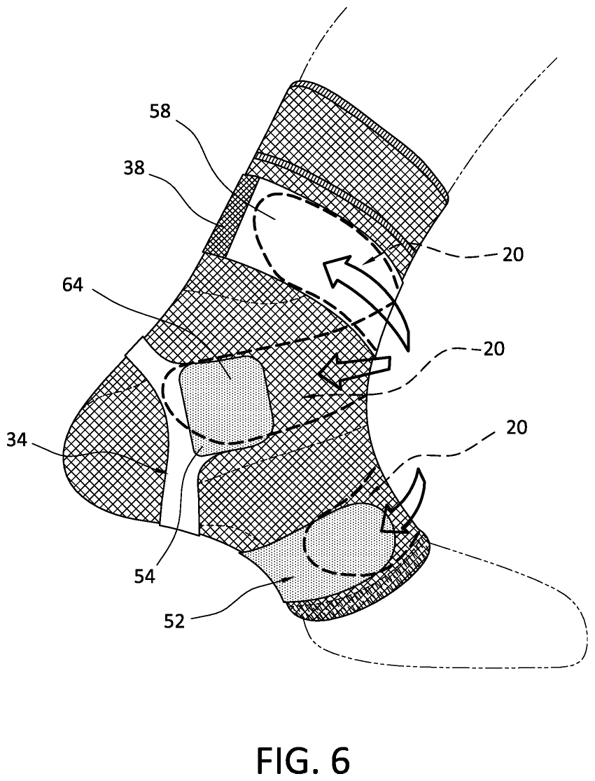

The strap system 14 mimics taping and provides versatility that enables a user to repeatedly don and secure the ankle support 10 without assistance from a clinician. The strap system 14 has versatility in the manner in which it is secured to the user's ankle in that it can be attached to at least two, preferably three, points after wrapping about a user's ankle, as exemplified in FIG. 6. The configuration of the strap system 14 makes donning consistent, provides tibia/fibula compression, and locks bones of the ankle in place or minimizes their movement by providing inversion/eversion.

As discussed in the foregoing, the strap system 14 employs segments that are elastic and inelastic to provide appropriate stretch and tension according to where the strap subassemblies 16, 18, 20 match or correspond to the bones and ligaments of an ankle. While segments of the strap system 14 are freely moveable and attachable relative to the sleeve 12, some segments are fixably secured to the sleeve 12 in predetermined places to arrest and offer anchored strapping. The combination of these elastic and inelastic segments increases strapping options for targeted support and comfort in a way that is customizable to a user's dimensions, activities, and dynamic needs.

The ankle support 10 may include visual cues and landing areas for placement of elements of the strap system 14 to assure the strap system 14 is secured in a consistent and effective manner. The landing areas may be optionally employed according to the treatment regime required for the user and may likewise serve as reinforcement areas or areas that inhibit stretching of the sleeve 12.

Returning to the strap system 14, the first, second and third strap subassemblies 16, 18, 20 preferably join at the junction 64 generally located on an anterior side of the ankle support 10 along a dorsal aspect Do thereof. The strap system 14, which comprises a plurality of strap subassemblies 16, 18, 20, may be continuously formed such that the first, second and third strap subassemblies 16, 18, 20 are secured to one another to form a single body comprising the strap subassemblies.

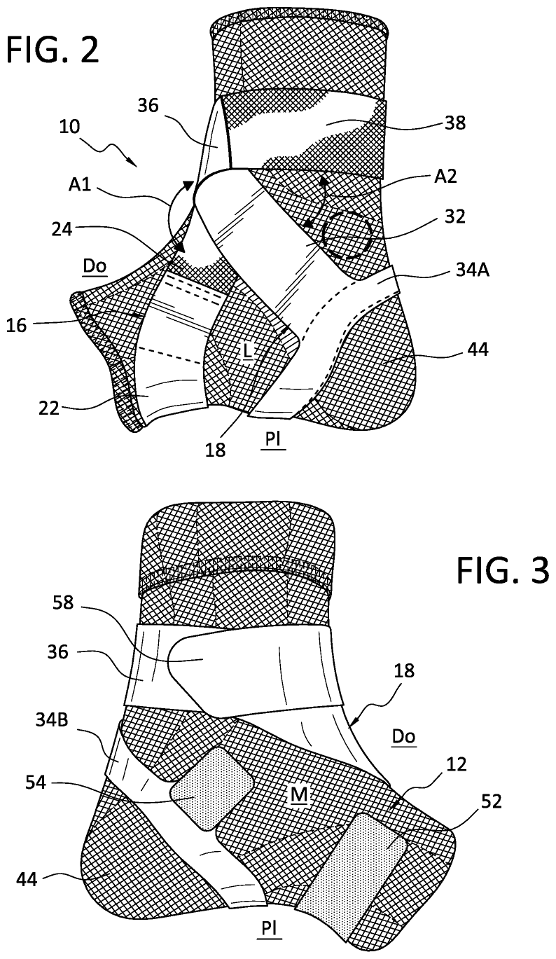

As shown in FIGS. 1 and 2, the first strap subassembly 16 is on or extends over a forefoot portion of the sleeve 12. The first strap subassembly 16 may be arranged obliquely relative to the second strap subassembly 18, to accommodate the shape of the foot and ankle anatomy. The first strap subassembly 16 may define a first inelastic segment 22 secured to the sleeve 12. The first strap subassembly 16 further defines an elastic segment 24 extending from the first inelastic segment 22 and securing to the third strap subassembly 20. The elastic segment 24 is provided to adjust to the user's foot anatomy.

The first inelastic segment 22 has a first portion 23 that may have an entirety of its length secured onto the sleeve 12 and may be stitched onto the sleeve 12. The sleeve 12 has a seam 66 on a posterior side P and/or plantar P1 aspect of the sleeve 12 and generally along the median plane, so the first portion 23 may extend from the seam 66 toward a dorsal side Do of the sleeve 12. The seam 66 may extend from proximal to distal edges 60, 62 dividing between medial and lateral sides M, L of the ankle support 10. A second portion 25 of the first inelastic segment 22 may extend freely from the first portion 23 and the sleeve 12.

In embodiments, a first end of the elastic segment 24 is secured to the third strap subassembly 20 and a second end is preferably secured to the second portion 25 of the first inelastic segment 22. The elastic segment 24 may be arranged obliquely relative to the third strap subassembly 20 at angle Al to provide a more anatomical transition to the third strap subassembly 20, particularly over the dorsal side Do of the ankle support 10. In this manner, the elastic segment 24 preferably extends over the dorsal side Do of the user's foot and the sleeve 12 to provide stretch, customization, and comfort when tensioning the strap system 14.

In embodiments such as shown in FIG. 1, the first strap subassembly 16 may further comprise a second inelastic segment 26. The second inelastic segment 26 may be attached at a first end to the third strap subassembly 20 and at a second end to the elastic segment 24. As with alternative embodiments and configurations, the arrangement of the second inelastic segment 26 between the elastic segment 24 of the first strap subassembly 16 and the third strap subassembly 20 may advantageously provide needed strength and structural stability while balancing a user's need for customizability and the need to provide an ankle support that is easy and comfortable to don. By arranging the elastic segment 24 between the first and second inelastic segments 22, 26, the first strap subassembly 16 may advantageously be configured to conform to a user's foot while providing immobilization or movement restriction in desired directions and locations.

As shown in FIGS. 3 and 6, the sleeve 12 includes a first or forefoot landing area 52 and a second or heel landing area 54. A tab 58 of the third strap subassembly 20 is adapted to secure to either of the forefoot and heel landing areas 52, 54, particularly shown in FIG. 6. The forefoot landing area 52 and the heel landing area 54 extend along the second or medial side M of the ankle support 10 and may be spaced apart by a distance. The forefoot landing area 52 may extend from the seam 66 of the sleeve 12 along the plantar side P1 of the forefoot region 43 or posterior side P, extending opposite from the first inelastic portion 23, and arranged along a median plane dividing the sleeve 12 along lateral L and medial sides M.

The strap system 14 includes a heel strap 34 secured to the sleeve 12. The midfoot or second strap subassembly 18 flexibly and freely extends from the heel strap 34. The second strap subassembly 18 may be entirely elastic or inelastic or may comprise first and second portions 28, 30, wherein a first portion 28 is generally inelastic and extends directly from the heel strap 34, and a second portion 30 is elastic and extends from the first portion 28 to the junction 64, serving a similar purpose to the elastic portion 24 of the first strap subassembly 16 in comfortably extending over the dorsal side Do of the user's foot and the sleeve 12. The elasticity in the second portion 30 may offer increased freedom of movement in the saggital place while the heel strap 34 is more rigid to stabilize the ankle in the coronal plane. In embodiments, the second portion 30 may be removed so the first portion 28 extends to the junction 64, since the elastic portion 24 in the forefoot strap may allow the second strap subassembly 18 to sufficiently adjust to the user's foot anatomy, activities, and dynamic needs, for example during different stages of rehabilitation.

The heel strap 34 preferably circumferentially extends about a heel portion 44 of the sleeve 12 to form a ring about the heel portion 44. The heel strap 34 extends about medial and lateral M, L sides of the ankle support 10 and has medial and lateral heel strap sections 34A, 34B. The medial and lateral heel strap sections 34A, 34B may be divided by the seam 66, which may extend along an entirety of the plantar and posterior portions of the sleeve 12.

The heel strap 34 may be fixedly secured to the sleeve 12, such as by stitching, welding or other means to maintain it in a fixed position without movement according to tensioning by the strap system 14. The entire heel strap 34 may be stitched to the sleeve 12. The heel strap 34 may be substantially more inelastic than the sleeve 12 so it does not yield or stretch once in place about a user's ankle. As an alternative to the heel strap 34, the heel portion 44 of the textile panel or sleeve 12 may have zones of inelasticity that mimic that support that the inelastic heel strap 34 provides.

The heel strap 34 is preferably inelastic. The heel strap 34 is at least less elastic than the surrounding knit of the sleeve 12, so it engages the heel and provides support. The midfoot strap of the second strap subassembly 18 is also inelastic for the same reason if the second portion 30 is omitted.

In embodiments, the strap system 14 is mostly inelastic except for two portions: the elastic segment 24 of the first strap subassembly 16 to adjust to anatomy, and an elastic segment 38 of the third strap subassembly 20 which wraps around the ankle for improved comfort. It has been found that if the strap system 14 were wholly inelastic, there would be increased risk of tourniquet and other discomfort. The strap system 14 would also lack generally the customizability offered by embodiments of the disclosure.

In other embodiments, such as the embodiment of FIG. 1, the second strap subassembly 18 may comprise a second inelastic segment 32. The second inelastic segment 32 may have a first end attaching to the third strap subassembly 20 generally proximate the junction 64 and a second end attaching to the elastic segment 30. As with the first strap subassembly 16 of embodiments of the present disclosure, the second strap subassembly 18 in this manner may be configured to comfortably accommodate and wrap around the user's leg and ankle according to the user's dimensions, activities, and dynamic needs, providing an enhanced degree of customizability while retaining the immobilization and stability benefits of traditional bracing solutions. The strap system 14 can be adapted with different elastic and inelastic segments from those discussed herein and are considered to fall within the scope of the disclosure.

The medial and lateral heel strap sections 34A, 34B may each form a generally semi-circular shape having first and second ends extending and attaching to the seam 66, whereby the seam 66 separates the medial and lateral heel strap sections 34A, 34B. The heel strap 34 may serve as a foundation or anchor from which the strap system 14 tensions about the sleeve 12, with the second strap subassembly 18 extending nearly perpendicularly away from the heel strap 34 toward the dorsal side Do of the ankle support 10. The first portion 28 of the second strap subassembly 18 may be formed from part of the heel strap 24, particularly on the lateral heel strap section 34A, or may be separate from the lateral heel strap section 34A but placed adjacent and/or may be attached thereto.

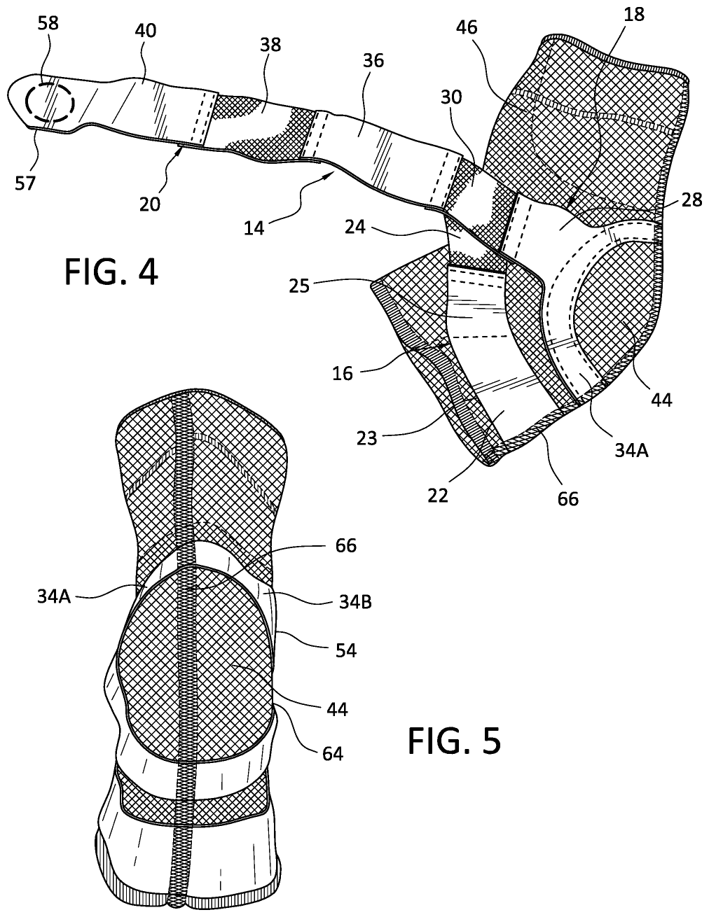

In the preferred embodiment of FIGS. 1-5, the second strap subassembly 18 joins the heel strap 34 on a lateral side L of the sleeve 12 such that the second strap subassembly 18 extends from the heel strap 34 toward a medial side M of the sleeve 12. The first and second strap subassemblies 16, 18 may be spaced by a distance D1 over and along the sleeve 12. The arrangement of the heel strap 34 with the second strap subassembly 18 provides enhanced locking and control around a user's foot, especially as the ankle support 10 is donned.

As shown in FIGS. 3 and 6, the heel strap 34 defines the second landing area 54 extending toward an anterior side A of the sleeve 12 from the medial heel strap 34B. The second landing area 54 may be wider than a width of the medial heel strap 34B and may extend obliquely therefrom toward a dorsal aspect Do of the sleeve 12. The second landing area 54 may extend in a similar manner from which the second strap subassembly 18 extends from the lateral heel strap 34A on the lateral side L of the ankle support 10. The tab 58 located at a free end of the third strap subassembly 20 is engageable with the second landing area 54 by extending over the dorsal aspect Do of the foot.

The second strap subassembly 18 may extend at an oblique angle A2 relative to the third strap subassembly 20, and from a heel portion 44 of the sleeve 12. The second strap subassembly 18 preferably extends from the heel portion 44 to the third strap subassembly 20 and is adapted to secure above the ankle and provide a space between the second and third strap subassemblies 18, 20 whereat a malleolus portion 48 is defined by the sleeve 12.

The third strap subassembly 20 preferably has a first inelastic segment 36 connected to the first and second strap subassemblies 16, 18 and extends toward the dorsal aspect Do of the sleeve 12. The elastic segment 38 of the third strap subassembly extends from the first inelastic segment 36. The third strap subassembly 20 has a second inelastic segment 40 extending from the elastic segment 38. The second inelastic segment 40 has the free end 57 and includes the tab 58 adapted to secure to the second inelastic segment 40. The separate segments 36, 38, 40 of the third strap subassembly 20 may be attached to each other in any suitable configuration, including releasably attached such as with hook and loop fastener, or permanently attached such as by stitching, adhesives, welding, or any other suitable manner.

The tab 58 and the first inelastic segment 36 may form a hook-and-loop fastener system. The tab 58 can secure with the first and second landing areas 52, 54, which form the same hook-and-loop fastener system. As illustrated by positions 1, 2, 3 as shown in FIG. 6, the inelastic segments of the strap system 14 may be configured so the tab 58 can secure to any of the inelastic segments of the strap system 14, thereby offering multiple contact and securing points for the strap system 14 according to specific needs of a user for protecting and/or rehabilitating an ankle or restricting motion of the ankle.

In use, the sleeve 10 is donned over the user's foot and ankle, and is appropriately aligned with the heel strap 34 secured above and around the user's heel. The strap system 14 is then appropriately secured, with the third strap assembly 20 extending around and above the lower leg, and then being secured onto itself, or one of the first and second landing areas 52, 54.

By providing an ankle support and methods for using the same according to the disclosed embodiments, the problems of existing ankle support devices providing ineffective, cumbersome, non-customizable, and inconsistent bracing and support of an ankle are addressed. The ankle support embodiments of the present disclosure advantageously provide a lightweight, breathable, and/or compressive sleeve, a versatile strap system, and/or a heel strap, which enable the ankle support embodiments to comfortably accommodate a user's dynamic needs and provide for more consistent and compliant usage.

While the disclosure discusses embodiments for the ankle, orthopedic device embodiments of the disclosure may be used with other limbs, joints and anatomical portions including the torso, shoulder, elbow, wrist/hand, hip, knee, and foot/ankle.

Not necessarily all such objects or advantages may be achieved under any embodiment of the disclosure. Those skilled in the art will recognize that the disclosure may be embodied or carried out to achieve or optimize one advantage or group of advantages as taught without achieving other objects or advantages as taught or suggested.

The skilled artisan will recognize the interchangeability of various components from different embodiments described. Besides the variations described, other known equivalents for each feature can be mixed and matched by one of ordinary skill in this art to construct an ankle support or an orthopedic brace utilizing the ankle support under principles of the present disclosure. Therefore, the embodiments described may be adapted to orthopedic systems for securing, supporting or comforting limbs or other anatomy.

Although ankle brace has been disclosed in certain preferred embodiments and examples, it therefore will be understood by those skilled in the art that the present disclosure extends beyond the disclosed embodiments to other alternative embodiments and/or uses of the disclosure and obvious modifications and equivalents. It is intended that the scope of the present disclosure disclosed should not be limited by the disclosed embodiments described above but should be determined only by a fair reading of the claims that follow.

1. An ankle support, comprising:
a sleeve;
a strap system having a heel strap secured to the sleeve, a first strap subassembly anchored to and extending from a first side of the sleeve, and a second strap subassembly anchored to and extending from the first side of the sleeve and spaced apart from the first strap subassembly.

2. The ankle support of claim 1, wherein the heel strap is inelastic.

3. The ankle support of claim 1, wherein the heel strap circumferentially extends about a heel portion of the sleeve to form a ring about the heel portion, and extends about medial and lateral sides of the ankle support, the heel strap having medial and lateral heel strap sections.

4. The ankle support of claim 1, wherein the sleeve is formed from a knitted fabric.

5. The ankle support of claim 4, wherein the heel strap is fixedly secured to the knitted fabric of the sleeve.

6. The ankle support of claim 4, wherein the heel strap is substantially more inelastic than the knitted fabric of the sleeve.

7. The ankle support of claim 1, wherein the sleeve defines first and second elasticity gradients having different elasticities from one another.

8. The ankle support of claim 1, wherein the sleeve defines a seam extending from proximal to distal edges dividing between medial and lateral sides of the ankle support, the seam dividing the heel strap into medial and lateral strap sections.

9. The ankle support of claim 8, wherein the medial and lateral strap sections each form a generally semi-circular shape having first and second ends extending to the seam.

10. The ankle support of claim 1, wherein the second strap subassembly joins the heel strap on a lateral side of the sleeve, the second strap subassembly extends from the heel strap toward a medial side of the sleeve.

11. The ankle support of claim 8, wherein the heel strap defines a first landing area extending toward an anterior side of the sleeve from the medial strap section.

12. The ankle support of claim 11, wherein the first landing area is wider than a width of the medial strap section and extends obliquely therefrom toward a dorsal aspect of the sleeve.

13. The ankle support of claim 12, wherein the strap system has a tab at a free end thereof that is engageable with the first landing area by extending over the dorsal aspect.

14. The ankle support of claim 1, wherein a first segment of the second strap subassembly extends from the heel strap, the first segment being inelastic and freely extending from the heel strap to extend over an anterior side of the sleeve to a medial side of the sleeve from the heel strap.

15. The ankle support of claim 14, the second strap subassembly extends from the lateral side of the sleeve and directly from a lateral heel strap section of the heel strap.

16. An ankle support having first and second sides divided by a median plane, the ankle support comprising:
a sleeve;
a strap system having a first strap subassembly anchored to and extending from the first side of the sleeve, a second strap subassembly anchored to and extending from the first side of the ankle support and spaced apart from the first strap subassembly, the first and second strap subassemblies joining at a third strap subassembly, the third strap subassembly being elongate and having a free end securable to at least one of the first, second or third strap subassemblies and wrappable about the sleeve.

17. The ankle support of claim 16, wherein the first, second and third strap subassemblies join at a junction generally located on an anterior side of the ankle support along a dorsal aspect thereof

18. The ankle support of claim 16, wherein the first strap subassembly is arranged obliquely relative to the second strap subassembly, wherein the first strap subassembly defines a first inelastic segment secured to the sleeve.

19. The ankle support of claim 18, wherein the first strap subassembly defines an elastic segment extending from the first inelastic segment and securing to the third strap subassembly, a first portion of the first inelastic segment having an entirety of its length secured onto the sleeve.

20. An ankle support having first and second sides divided by a median plane, the ankle support comprising:
a sleeve;
a strap system having a first strap subassembly anchored to and extending from the first side of the sleeve, a second strap subassembly anchored to and extending from the first side of the sleeve and spaced apart from the first strap subassembly, the first and second strap subassemblies joining at a third strap subassembly, the third strap subassembly being elongate and having a free end securable to at least one of the first, second or third strap subassemblies and wrappable about the sleeve;
wherein the first, second and third strap subassemblies join at a junction generally located on an anterior side of the ankle support along a dorsal aspect thereof;
wherein the first strap subassembly is arranged obliquely relative to the second strap subassembly, wherein the first strap subassembly defines a first inelastic segment secured to the sleeve and a second inelastic segment having a first end secured to the third strap subassembly and a second end secured to an elastic segment;
wherein the third strap subassembly has a first inelastic segment connected to the first and second strap subassemblies and extending toward the dorsal aspect of the sleeve and an elastic segment extending from the first inelastic segment;
wherein the second strap subassembly extends from a heel portion to the third strap subassembly and is adapted to be secured above the ankle and to provide a space between the second and third strap subassemblies whereat a malleolus portion is defined by the sleeve, the second strap subassembly extending at an oblique angle relative to the third strap subassembly.
